(19) United States
(12) Patent Application Publication
(10) Pub. No.: US 2010/0158836 A1
(43) Pub. Date: Jun. 24, 2010

(54) Method of Coloring Nails and a Nail Polish Formulation

(76) Inventors: Cindy Nelson, Los Angeles, CA (US); Julia Palmer, Los Angeles, CA (US)

Correspondence Address: MARC E. HANKIN, ESQ., 11414 THURSTON CIRCLE, LOS ANGELES, CA, 90049, US

(21) Appl. No.: 12/338,948

(22) Filed: Dec. 18, 2008

(51) Int. Cl. A61K 8/30; A45D 29/00; A61Q 3/02

(52) U.S. Cl. 424/61; 132/200

(57) ABSTRACT

Various embodiments of this invention discloses a translucent and colored nail polish glaze, a nail polish base, and a method for combining nail polish base colors with translucent and colored nail polish glazes to create a multiple of different colors and color shades from a minimal number of nail polish base and glaze colors.

### BACKGROUND OF THE INVENTION

This invention generally relates to liquid nail polish formulations and methods for applying the nail polish formulations to human nails as a colored coating. Specifically, it pertains to a translucent and colored nail polish glaze, a nail polish base, and a method for combining nail polish base colors with translucent and colored nail polish glazes to create a multiple of different colors and color shades from a minimal number of nail polish base and glaze colors.

Nail polish is an important part of creating an interesting and made-up look. Nail polish, in a variety of formulations and colors, has been around for centuries. However, the typical nail polish is opaque and thus, even if a user coats (in even layers) a nail with two separate nail polish colors, the nail will only display the color of the last nail polish applied. In this manner, a user can only color nails in a one to one ratio with the number of nail polish colors. Specifically, if the user has two nail polish colors, the user can color the nails in two colors. Thus, the typical nail polish does not allow the user to create a multiple number of nail colors from the nail polish colors.

Nail polish glaze is a top coat for a nail that goes over a base nail polish coat. Nail polish glaze is important for protecting the nail polish base coat and extending the duration of the color imparted by the nail polish base coat. Nail polish glaze is currently only available as a clear or transparent coating. This clear coating allows the base nail polish color to be completely visible and the color does not change at all as seen through the clear glaze.

Despite centuries of availability of nail polish base colors, nail polish glazes, and systems and methods for coloring nails, there has never before been disclosed a translucent nail polish glaze formulation that allows the user to alter the color of a nail (in multiple ways) that already has a base nail polish coat. Thus, there is a need in the art for a translucent and color altering nail polish glaze and a method for combining nail polish base colors with translucent and colored nail polish glazes to create a multiple of different colors and color shades.

### BRIEF SUMMARY OF THE INVENTION

To minimize the limitations in the prior art, and to minimize other limitations that will become apparent upon reading and understanding the present specification, the present invention discloses a translucent and colored nail polish glaze, a nail polish base, and a method for combining nail polish base colors with translucent and colored nail polish glazes to create a multiple of different colors and color shades from a minimal number of nail polish base and glaze colors.

One embodiment of this invention is a translucent nail polish glaze formulation comprising: ethyl acetate; butyl acetate; isopropyl alcohol; nitrocellulose; one or more chemicals selected from the group consisting of adipic acid, neopentyl glycol, trimellitic anhydride copolymer; trimethyl pentanyl diisobutyrate; triphenyl phosphate; stearalkonium hectorite; diacetone alcohol; citric acid; dimethicone; and benzophenone-1. The translucent nail polish glaze further comprising one or more chemicals selected from the group consisting of: nacreous pigments; red no. 6 barium lake, red no. 7 calcium lake, red no. 34 calcium lake, yellow no. 5 aluminum lake, ferric (ammonium) ferrocyanide, and violet no. 2. The opaque nail polish glaze further comprising one or more chemicals selected from the group consisting of: nacreous pigments; stearalkonium hectorite, titanium dioxide, mica, bismuth oxychloride, talc, iron oxides (red and black), red no. 6 barium lake, red no. 7 calcium lake, red no. 34 calcium lake, yellow no. 5 aluminum lake, ferric (ammonium) ferrocyanide, violet no. 2, acrylates copolymers, polyethylene terephthalate, tosylamide, epoxy resin, N-butyl alcohol, SD Alcohol 40B, camphor and SD Alcohol 3-A.

Another embodiment of the invention is an opaque nail polish base comprising: ethyl acetate; butyl acetate; isopropyl alcohol; nitrocellulose; one or more chemicals selected from the group consisting of, adipic acid, neopentyl glycol, trimellitic anhydride copolymer; trimethyl pentanyl diisobutyrate; triphenyl phosphate; stearalkonium hectorite; diacetone alcohol; citric acid; dimethicone; and benzophenone-1. The opaque nail polish base further comprising one or more chemicals selected from the group consisting of: nacreous pigments; stearalkonium hectorite, titanium dioxide, mica, bismuth oxychloride, talc, iron oxides (red and black), red no. 6 barium lake, red no. 7 calcium lake, red no. 34 calcium lake, yellow no. 5 aluminum lake, ferric (ammonium) ferrocyanide, and violet no. 2.

Another embodiment of the invention is a method of coloring the nails of a human comprising: providing one or more color charts that describes an one or more steps to create one or more resulting colors that result from combining one or more nail polish bases with one or more nail polish glazes and one or more nail polish special effects; determining what a final color of one or more nails will be by consulting the one or more color charts; following the one or more steps to create the determined final color of the one or more nails; selecting a nail polish base color; applying the nail polish base color to an one or more nails; selecting a first nail polish glaze color; applying the first nail polish glaze color to the one or more nails, wherein the first nail polish glaze color is applied only to the one or more nails with the nail polish base color, wherein the first nail polish glaze color is translucent, wherein a combination of the first nail polish glaze color, which covers in a layered manner the nail polish base color on the one or more nails, causes the one or more nails to have a third color that is different from the nail polish glaze color and the nail polish base color; applying the first nail polish glaze color a second time to the one or more nails, wherein the second application of the first nail polish glaze color causes the one or more nails to have a fourth color that is different from the third color, the nail polish glaze color, and the nail polish base color, wherein the fourth color is a deeper color shade than the third color.

Another embodiment of the invention includes applying a second nail polish glaze color instead of a second coat of the same nail polish glaze color, wherein the second nail polish glaze color is also translucent.

Another embodiment of the invention is the method of coloring the nails of a human, further comprising applying a first nail polish special effect. The special effect should be applied after applying the nail polish base color, but it can be applied anytime thereafter, including: 1) before or after the application of the first nail polish glaze color; 2) before or after the second application of the first nail polish; or before or after the application of the second nail polish glaze. The method may also include the step of applying a first nail polish special effect a second time (at anytime after the first application of the first special effect) or the step of applying a second nail polish special effect (at anytime after the application of the first nail polish special effect).

An object of this invention is to provide a colored nail polish base that is available in many colors.

Another object of this invention is to provide a colored translucent nail polish glaze or top coat that is available in many colors.

Another object of this invention is to provide a method for combining a nail polish base with a colored translucent nail polish glaze to create new colors.

Another object of this invention is to provide a way to create a multiple of different colors from the combination of just a few nail polish base colors and nail polish glaze colors.

Another object of this invention is to provide a nail polish application method that offers flexibility and individuality.

Another object of this invention is to provide a color scheme for polishing nails that is based from the world of gems, precious stones, and the artist color wheel.

Another object of this invention is to provide a color scheme for polishing nails that allows a user to create everything from brilliant colors, deep vamps, smoky tones, special effects, and creamy colors.

Another object of this invention is to provide a nail polish base and glaze that dries faster than standard nail polish, and involves the same brush-on application steps as standard nail polish. The nail polish base and glaze of the present invention provide long durable wear and the ability to refresh, intensify or change the color or effect completely.

Another object of the invention is to provide a nail polish where all color and effects combinations have depth and an intense shine.

Another object of the invention is to provide an affordable and cost effective nail polishing system.

Another object of the invention is to provide a nail polish glaze that can be used with currently available standard nail polishes.

An object of the present invention is to provide an apparatus and method that will overcome the deficiencies of the prior art.

Other features and advantages are inherent in the translucent and color altering nail polish glaze, nail polish base, and a method for combining nail polish base colors with translucent and colored nail polish glazes to create a multiple of different colors and color shades.

### BRIEF DESCRIPTION OF THE DRAWINGS

FIG. 1 is an illustration of a color chart of one embodiment of the method of coloring nails.

FIG. 2 is an illustration of a color chart of one embodiment of the method of coloring nails and shows the application of two different nail polish glazes and lists the nail polish base colors and nail polish glaze colors with fanciful names.

FIG. 3 is an illustration of a color chart of one embodiment of the method of coloring nails and shows the application of a nail polish special effect.

FIG. 4 is an illustration of a color chart of one embodiment of the method of coloring nails with pastel colors.

FIG. 5 is an illustration of a color chart of one embodiment of the method of coloring nails and shows the application of two coats of a nail polish special effect.

FIG. 6 is an illustration of a color chart of one embodiment of the method of coloring nails and shows the application of two different nail polish special effects.

### DETAILED DESCRIPTIONS OF THE DRAWINGS

In the following detailed description of the preferred embodiment, reference is made to the accompanying drawings that form a part hereof, and in which is shown, by way of illustration, a specific embodiment in which the invention may be practiced. It is to be understood that other embodiments may be utilized and structural changes may be made without departing from the scope of the present invention.

In the following detailed description of various embodiments of the invention, numerous specific details are set forth in order to provide a thorough understanding of various aspects of one or more embodiments of the invention. However, one or more embodiments of the invention may be practiced without these specific details. In other instances, well-known methods, procedures, and/or components have not been described in detail so as not to unnecessarily obscure aspects of embodiments of the invention.

The nail polish formulation of the present invention is preferably made from Butyl Acetate, Ethyl Acetate, Isopropyl Alcohol, Nitrocellulose, Adipic Acid/Neopentyl Glycol/Trimellitic Anhydride Copolymer, Trimethyl Pentanyl Diisobutyrate, Triphenyl Phospate, Stearalkonium Hectorite, Diaceton Alcohol, Citric Acid, Dimethicone, and Benzohenone-1.

The preferred function and the preferred % by weight of each ingredient of the typical nail polish formulation of the present invention is:

| Ingredient | %/Weight | Function |
|---|---|---|
| Ethyl Acetate | 27-34 | Solvent |
| Butyl Acetate | 24-29 | Solvent |
| Isopropyl Alcohol | 15-20 | Solvent |
| Nitrocellulose | 9-11 | Film Former |
| Adipic Acid/Neopentyl | 6-7 | Film Former |
| Glycol/Trimellitic | | |
| Anhydride Copolymer | | |
| Trimethyl Pentanyl Diisobutyrate | 3-4 | Plasticizer |
| Triphenyl Phosphate | 3-4 | Plasticizer |
| Stearalkonium Hectorite | 1-2 | Suspending Agent |
| Diacetone Alcohol | 0.73-0.83 | Solvent |
| Citric Acid | 0.03-0.05 | pH Adjuster |
| Dimethicone | 0.03-0.05 | Conditioning Agent |
| Benzophenone-1 | 0.03-0.05 | UV Absorber |

The solvents act as the base liquid into which all of the other ingredients are mixed to form the solution of the present invention. Plasticizers are additives that increase the plasticity or fluidity of the material to which they are added. A plasticizer for plastics typically softens the final product increasing its flexibility. A film former is any substance that will change from liquid to solid in such a manner as to form a film over a surface. A suspending agent helps the ingredients of the solution stay suspended within the solution and, thus, prevents caking at the bottom. One of the properties of the nail polish base and glaze formulations of the present invention is that they can be easily re-suspended by the use of moderate agitation.

If a nail polish base formulation is desired one or more of the following colorants are added: Nacreous Pigments, Stearalkonium Hectorite, Titanium Dioxide, Mica, Bismuth Oxychloride, Talc, Iron Oxides (red and black), Red No. 6 Barium Lake, Red No. 7 Calcium Lake, Red No. 34 Calcium Lake, Yellow No. 5 Aluminum Lake, Ferric (Ammonium) Ferrocyanide, and Violet No. 2. These colorants are added to give the nail polish base its color and/or opaque property.

A nail polish base is created by combining the film formers, plasticizers, pH adjusters, conditioners, UV absorbers, and suspending agents into the solvents. The colorant ingredients are then added to give the nail polish base a final color. For example, Ferric Ammonium Ferrocyanide, Yellow No. 5, and Mica are added to create the color green (also known as “anxious,” as described below).

The nail polish base formulation described above preferably results in a quick drying and opaque nail polish. Depending on what colorants are added, the nail polish base formulation results in a wide array of striking and vibrant colors.

If a translucent nail polish glaze formulation is desired one or more of the following colorants are added: Nacreous Pigments, Red No. 6 Barium Lake, Red No. 7 Calcium Lake, Red No. 34 Calcium Lake, Yellow No. 5 Aluminum Lake, Ferric (ammonium) Ferrocyanide, and Violet No. 2. These additional ingredients give the translucent nail polish glaze its color.

Importantly, no ingredients that cause significant or complete opaqueness are added to the nail polish glaze formulation.

The translucent nail polish glaze is created in essentially the same way as the nail polish base (as described above), but the nail polish glaze is translucent and allows the base color to be seen through the filter of the nail polish glaze that is added on top of the nail polish base.

Other embodiments of the nail polish glaze formulation create nail polish special effect glazes (also called nail polish special effects). Nail polish special effects include black (Onyx), dark gray (Charcoal or Moonstone), light gray, white, shiny or reflective (Minor), gold glitter (or sparkle), and silver glitter (or sparkle). Nail polish special effects may have alternative formulations from the basic nail polish glaze.

For example, reflective (Mirror) nail polish special effect has the formulation of:

| Ingredient | %/Weight | Function |
|---|---|---|
| Ethyl Acetate | 24-29 | Solvent |
| SD Alcohol 40B | 20-25 | Solvent |
| Butyl Acetate | 17-21 | Solvent |
| Nitrocellulose | 9-11 | Film Former |
| Adipic Acid/Neopentyl | 5-7 | Film Former |
| Glycol/Trimellitic | | |
| Anhydride Copolymer | | |
| SD Alcohol 3-A | 4-5 | Solvent |
| N-Butyl Alcohol | 3-4 | Solvent |
| Trimethyl Pentanyl Diisobutyrate | 2-3 | Plasticizer |
| Triphenyl Phosphate | 2-3 | Plasticizer |
| Camphor | 0.40-0.50 | Plasticizer |
| Benzophenone-1 | 0.10-0.20 | UV Absorber |
| Violet No. 2 | qs | Colorant |

In another example, black (Onyx) nail polish special effect has the formulation of:

| Ingredient | %/Weight | Function |
|---|---|---|
| Ethyl Acetate | 27-34 | Solvent |
| Butyl Acetate | 24-29 | Solvent |
| Isopropyl Alcohol | 15-20 | Solvent |
| Nitrocellulose | 9-11 | Film Former |
| Adipic Acid/Neopentyl | 6-7 | Film Former |
| Glycol/Trimellitic | | |
| Anhydride Copolymer | | |
| Trimethyl Pentanyl Diisobutyrate | 3-4 | Plasticizer |
| Triphenyl Phosphate | 3-4 | Plasticizer |
| Stearalkonium Hectorite | 1-2 | Suspending Agent |
| Diacetone Alcohol | 0.73-0.83 | Solvent |
| Citric Acid | 0.03-0.05 | pH Adjuster |
| Dimethicone | 0.03-0.05 | Conditioning Agent |
| Benzophenone-1 | 0.03-0.05 | UV Absorber |
| Black Iron Oxide | qs | Colorant |

The black nail polish special effect is translucent and gives underlying colors a deep vamp look.

In another example, dark gray (Charcoal or Moonstone) nail polish special effect has the formulation of:

| Ingredient | %/Weight | Function |
|---|---|---|
| Ethyl Acetate | 27-34 | Solvent |
| Butyl Acetate | 24-29 | Solvent |
| Isopropyl Alcohol | 15-20 | Solvent |
| Nitrocellulose | 9-11 | Film Former |
| Adipic Acid/Neopentyl | 6-7 | Film Former |
| Glycol/Trimellitic | | |
| Anhydride Copolymer | | |
| Trimethyl Pentanyl Diisobutyrate | 3-4 | Plasticizer |
| Triphenyl Phosphate | 3-4 | Plasticizer |
| Stearalkonium Hectorite | 1-2 | Suspending Agent |
| Diacetone Alcohol | 0.73-0.83 | Solvent |
| Citric Acid | 0.03-0.05 | pH Adjuster |
| Dimethicone | 0.03-0.05 | Conditioning Agent |
| Benzophenone-1 | 0.03-0.05 | UV Absorber |
| Black Iron Oxide | qs | Colorant |
| Red Iron Oxide | qs | Colorant |
| Titanium Dioxide | qs | Colorant |

The gray nail polish special effect is translucent and gives underlying colors a dark smokey look.

In another example, silver glitter (Silver Sparkle) or gold glitter (Gold Sparkle) nail polish special effects have the formulation of:

| Ingredient | %/Weight | Function |
|---|---|---|
| Ethyl Acetate | 35-42 | Solvent |
| Butyl Acetate | 13-16 | Solvent |
| Nitrocellulose | 12-15 | Film Former |
| Triphenyl Phosphate | 8-9 | Plasticizer |
| Isopropyl Alcohol | 5-6 | Solvent |
| SD Alcohol 40B | 5-6 | Solvent |
| Tosylamide/Epoxy Resin | 5-6 | Film Former |
| Styrene Acrylates Copolymer | 2-3 | Film Former |
| Silica | 1-2 | Suspending Agent |
| Polyethylene Terephthalate | qs | Film Former/Polyester |
| | | Flake/Colorant) |
| Acrylates Copolymers | qs | Film Former/Polyester |
| | | Flake/Colorant) |

The silver and gold glitter nail polish special effect is translucent and gives the nail a three dimensional shine, sparkle, or glitter effect.

In another example, light gray nail polish special effect has the formulation of:

| Ingredient | %/Weight | Function |
|---|---|---|
| Ethyl Acetate | 27-34 | Solvent |
| Butyl Acetate | 24-29 | Solvent |
| Isopropyl Alcohol | 15-20 | Solvent |
| Nitrocellulose | 9-11 | Film Former |
| Adipic Acid/Neopentyl | 6-7 | Film Former |
| Glycol/Trimellitic | | |
| Anhydride Copolymer | | |
| Trimethyl Pentanyl Diisobutyrate | 3-4 | Plasticizer |
| Triphenyl Phosphate | 3-4 | Plasticizer |
| Stearalkonium Hectorite | 1-2 | Suspending Agent |
| Diacetone Alcohol | 0.73-0.83 | Solvent |
| Citric Acid | 0.03-0.05 | pH Adjuster |
| Dimethicone | 0.03-0.05 | Conditioning Agent |
| Benzophenone-1 | 0.03-0.05 | UV Absorber |
| Black Iron Oxide | qs | Colorant |
| Titanium Dioxide | qs | Colorant |

The light gray nail polish special effect is translucent and gives the nail a light smoky look.

In another example, white nail polish special effect has the formulation of:

| Ingredient | %/Weight | Function |
|---|---|---|
| Ethyl Acetate | 27-34 | Solvent |
| Butyl Acetate | 24-29 | Solvent |
| Isopropyl Alcohol | 15-20 | Solvent |
| Nitrocellulose | 9-11 | Film Former |
| Adipic Acid/Neopentyl | 6-7 | Film Former |
| Glycol/Trimellitic | | |
| Anhydride Copolymer | | |
| Trimethyl Pentanyl Diisobutyrate | 3-4 | Plasticizer |
| Triphenyl Phosphate | 3-4 | Plasticizer |
| Stearalkonium Hectorite | 1-2 | Suspending Agent |
| Diacetone Alcohol | 0.73-0.83 | Solvent |
| Citric Acid | 0.03-0.05 | pH Adjuster |
| Dimethicone | 0.03-0.05 | Conditioning Agent |
| Benzophenone-1 | 0.03-0.05 | UV Absorber |
| Titanium Dioxide | qs | Colorant |

The white nail polish special effect is translucent and gives the nail a creamy look.

The method of combining the above described nail polish base and nail polish glaze to create a multiple of colors is now described here in detail.

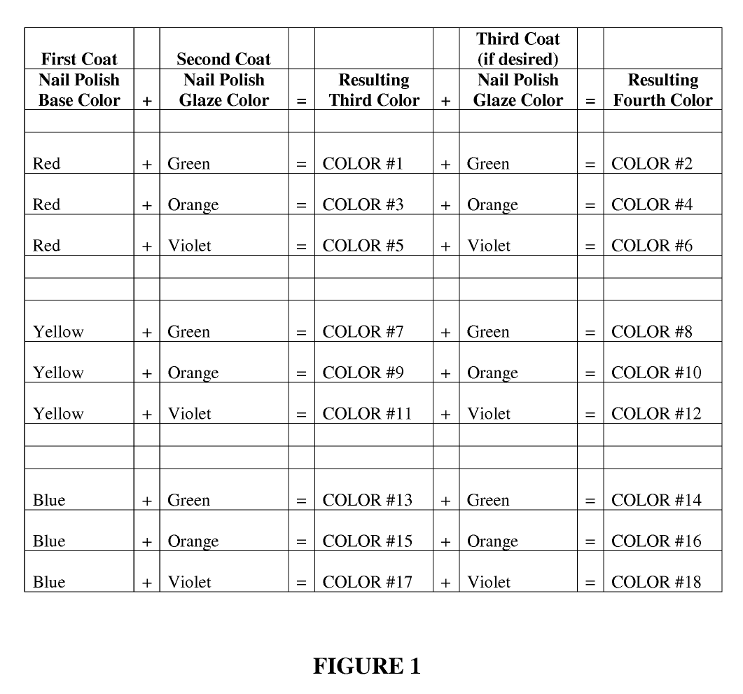

Preferably, the user of the nail polishing method of the present invention starts with a set number of bottles of nail polish bases, nail polish glazes, and nail polish special effects. Typically, the user will purchase a set of three bottles of base, three bottles of glaze, and one bottle of special effect. The bases, glazes, and special effects are preferably liquid and are applied to the nail with a brush in the same manner that a standard nail polish is applied. The sets of bases, glazes, and special effects will preferably come with a color chart and a color wheel. The color chart, which is shown in various embodiments in FIGS. 1 to 6, details resulting colors and outlines some of the steps (of the method of the present invention) that must be taken to achieve the resulting colors. The steps detail combining one or more of the glazes with one or more of the bases and with one or more of the special effects. The color wheel is simply a circular chart (similar to a clock face) that shows a representation of each of the resulting colors so that the user can see what the resulting color looks like before going through the process of coloring the nails.

The first step of the method of the present invention is the user (who already has the color wheel, color chart, and bottles of base, glaze, and special effect) consulting the color wheel and color chart (preferably a combined single page and colorized document) to review what resulting nail colors are available. The user then selects a resulting color. After a resulting color is selected, the user then follows the steps on the color chart to create the determined final resulting color.

The steps listed on the color chart recite the next steps of the method of the present invention. First, a nail polish base color is selected and that color is applied to one or more nails of the hands and/or feet of a human. After the base is dry, a first nail polish glaze color is selected and applied. Preferably, the glaze is only applied to those nails that have the base color on them. Preferably, the nail polish glaze is colored and translucent. The colored and translucent nature of the glaze allows the glaze to cover the nail polish base and alter the visible color of the nail so that it appears as a third color that is different from both the base color and the glaze color. Essentially, the base color is altered, in the eyes of a viewer, when looked at through the translucent filter of the nail polish glaze color.

The user can stop at the third color, or the user can continue the steps of the method of the current invention and apply a second coat of the first nail polish glaze. This second coat causes the nail to have a fourth color that is different from the third color, the nail polish glaze color, and the nail polish base color. Typically, this fourth color is simply a shade different from the third color and has a more intense and deeper level of color.

FIG. 1 is an illustration of a color chart of one embodiment of the method of coloring nails. Figure one shows a color chart that details the preferred steps of the method of applying a nail polish base and a nail polish glaze to create a third and fourth resulting color. As shown in FIG. 1 the typical set of polish bases and polish glazes comes with three colors of base and three colors of glaze. FIG. 1 illustrates one way of combining the six bottles to create eighteen different colors. FIG. 1 shows that the user has red, yellow, and blue nail polish base and green, orange, and violet nail polish glaze. FIG. 1 shows that with only six initial colors available to the user, the user is able to create at least three times as many colors.

Although only six colors are described in FIG. 1, the nail polish base, nail polish glaze, and method of the present invention may be practiced with any and all colors of the color spectrum. Although not shown in FIG. 1, the nail polish base colors and the nail polish glaze colors are preferably available in all the colors of an artist color wheel. The colors of a standard artist color wheel include red, red-orange, orange, orange-yellow, yellow, yellow-green, green, blue-green, blue, blue-violet, violet, and violet-red. If a user has twelve bottles of nail polish base and twelve bottles of nail polish glaze, in the colors of the artist color wheel, the user will be able to combine these colors, using the method of the current invention, to color the nails in hundreds of different resulting colors and color shades.

Another embodiment of the invention substitutes a second application of the first nail polish glaze for an application of a second nail polish glaze that is a different color from the first nail polish glaze. Preferably, this second nail polish glaze is also colored and translucent.

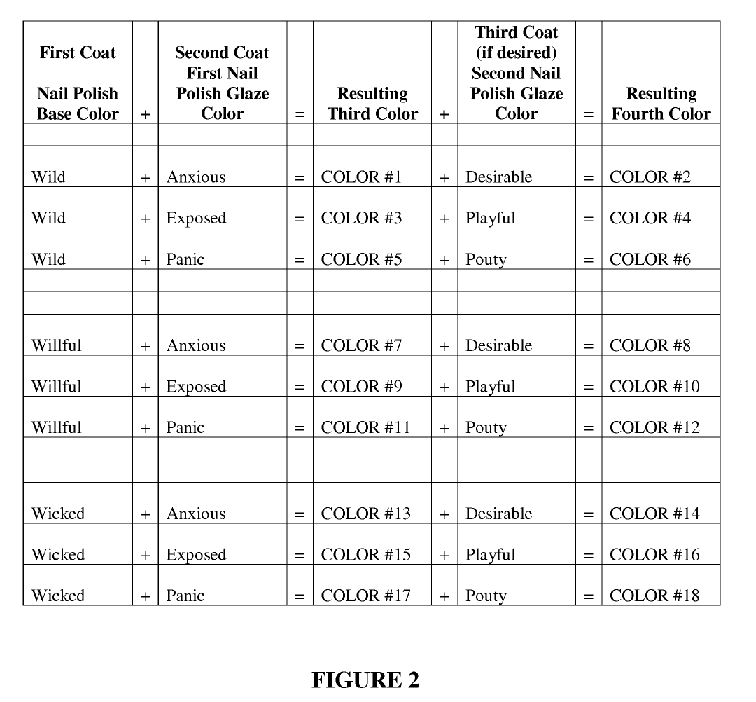

FIG. 2 is an illustration of a color chart of one embodiment of the method of coloring nails and shows the application of two different nail polish glazes and lists the nail polish base colors and nail polish glaze colors with fanciful names. As shown in FIG. 2, the second (yet still optional) glaze coat is a different color from the first glaze applied. This allows the user to create even more colors and color shades. FIG. 2 also shows how the colors of the nail polish bases and nail polish glazes are preferably presented with fanciful names. Although the fanciful names can be any interesting name, the preferred glaze names are as follows: wild (red); willful (yellow); wicked (blue); sassy (red-orange); skittish (orange); neurotic (yellow-orange); fierce (yellow-green); loco (blue-violet); lewd (violet); lust (red-violet); anguish (green); and angel (blue-green). The nail polish glazes also include additional nail polish special effects, which are discussed in detail below. In addition to bold color glazes the nail polish glazes can be shiny (reflective or mirrored), or they can be pastel colored.

The preferred base names are as follows: sultry (red); empowered (yellow); numb (blue); desirable (red-orange); exposed (orange); vivacious (yellow-orange); envy (yellow-green); pouty (blue-violet); panic (violet); playful (red-violet); anxious (green); and edgy (blue-green). In addition to being brightly colored, the bases can be more muted, such as a natural color, or they can be pastel colored. If a bright bold color is used as the base, the glaze is preferably also a bright bold color. Similarly, if a pastel color is used as the base, the glaze is preferably also a pastel color.

Another embodiment of the invention includes an additional step of applying a first nail polish special effect after applying the nail polish base color and before applying the first nail polish glaze color.

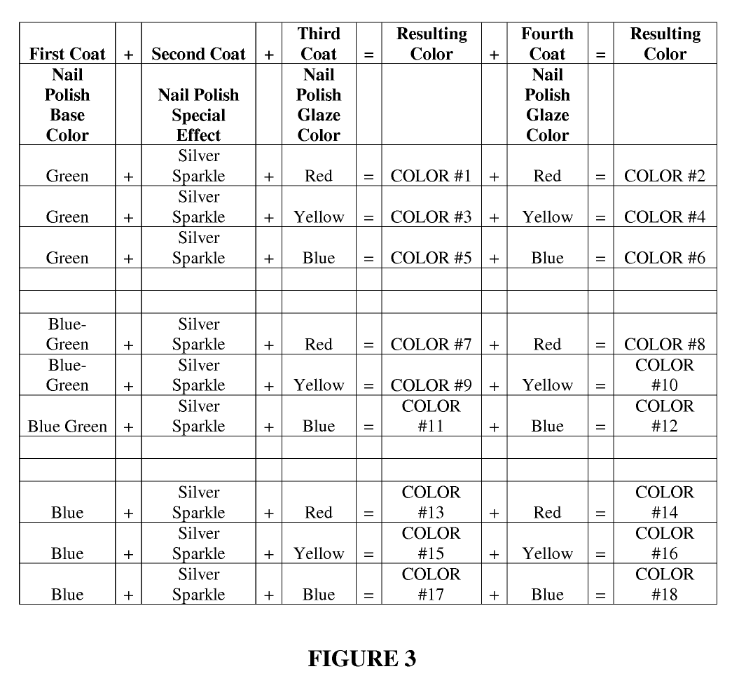

FIG. 3 is an illustration of a color chart of one embodiment of the method of coloring nails and shows the application of a nail polish special effect. As shown in FIG. 3 the special effect is preferably added before the first coat of glaze, but it can be applied at any time without deviating from the scope of the invention. Although the special effect disclosed in FIG. 3 is a silver sparkle, the special effect can be any visible light altering effect, such as a blackening or darkening agent (black or onyx), a dark graying agent (charcoal or moonstone), a light graying agent, a whitening agent, or a metallic sparkle agent (glitter) without deviating from the scope of the invention. The preferred names of the special effects are gold sparkle, silver sparkle, onyx, moonstone, light gray, and white. The nail polish special effects are preferably a modified version of the nail polish glaze and both are translucent. The nail polish special effects differ from the glazes because, rather than being a color of the artist color wheel, they are metallic, white, black, gray, light gray, or glitter containing. The nail polish special effects allow the user to create deep vamp colors, smoky colors, sparkling colors, or even creamy colors. Nails with the nail polish special effect will still have the same rich depth of color of nails without nail polish special effect, because the nail polish special effect is translucent.

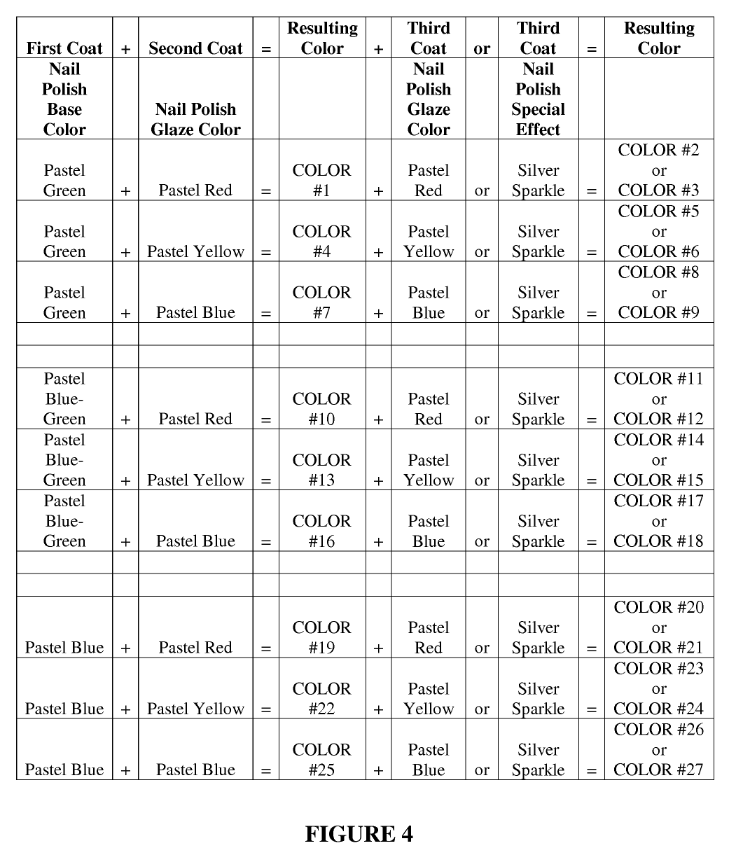

FIG. 4 is an illustration of a color chart of one embodiment of the method of coloring nails with pastel colors. As shown in FIG. 4 the first coat is a pastel color, and the second coat is a pastel nail polish glaze color. Although bold or bright colors can be used with pastels, this is not preferred because the colors do not look good mixed.

As shown in FIG. 4, the third coat can either be a second coat of the pastel nail polish glaze color or a nail polish special effect, such as silver sparkle. Although the sparkle special effect nail polishes are preferably applied to the nails before the regular nail polish glaze, this is not the case with pastels. Because the pastel glazes are not as translucent as the bold/bright glazes, putting a pastel glaze over a sparkle special effect glaze will simply not look good. Thus, if the user wishes to combine a sparkle special effect with a pastel base and a pastel glaze(s), the sparkle should be applied last. Finally, although FIG. 4 shows the addition of a second coat of the same nail polish glaze, a different (but preferably pastel) nail polish glaze may be applied without deviating from the scope of the invention.

Another embodiment of the invention includes the step of applying a first nail polish special effect to the nails after applying the first nail polish glaze color and before applying the second (and optional) application of the first nail polish glaze color. Alternatively, the method of the present invention can include applying the first nail polish special effect a second time in place of the second application of the first nail polish glaze.

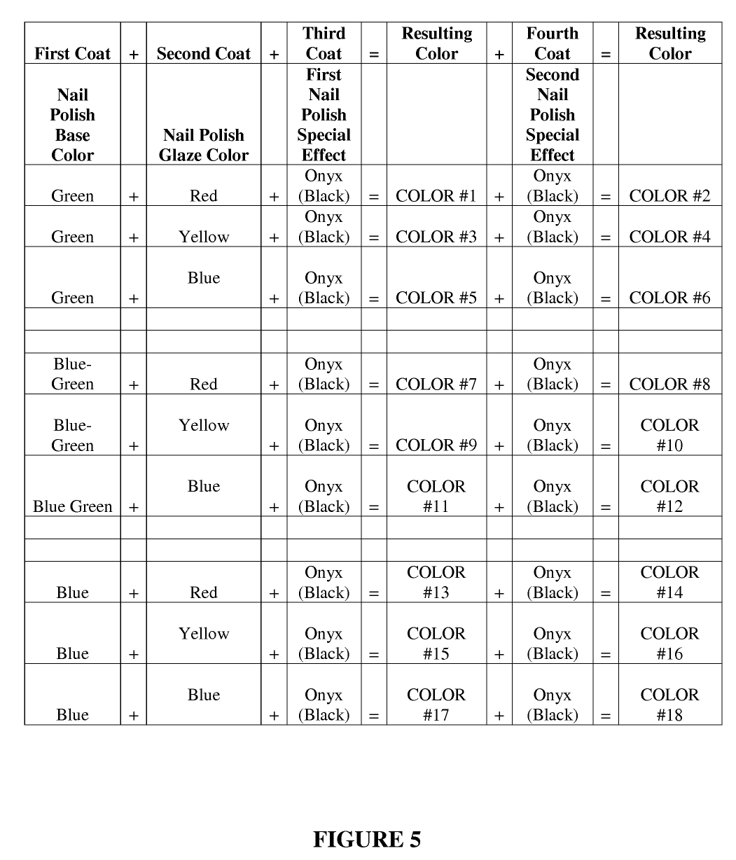

FIG. 5 is an illustration of a color chart of one embodiment of the method of coloring nails and shows the application of two coats of a nail polish special effect. As shown in FIG. 5 the Onyx special effect is applied after the first nail polish glaze color. Additionally, the optional final nail polish coat is a second application of the first nail polish special effect, in this case Onyx, in place of the second application of the first nail polish glaze.

Another embodiment of the method of the present invention involves applying a second nail polish special effect to the nails after applying the first nail polish special effect. The second nail polish special effect essentially replaces the second application of the first nail polish glaze.

Another embodiment of the method of the present invention includes the step of applying a first nail polish special effect to the nails after applying the first nail polish glaze color. Additionally, the first nail polish special effect can be applied a second time in addition to or in place of applying the second nail polish glaze color or the second application of the first nail polish glaze color. Finally, a second nail polish special effect, which is different from the first nail polish special effect, may be applied after the application of the first nail polish special effect.

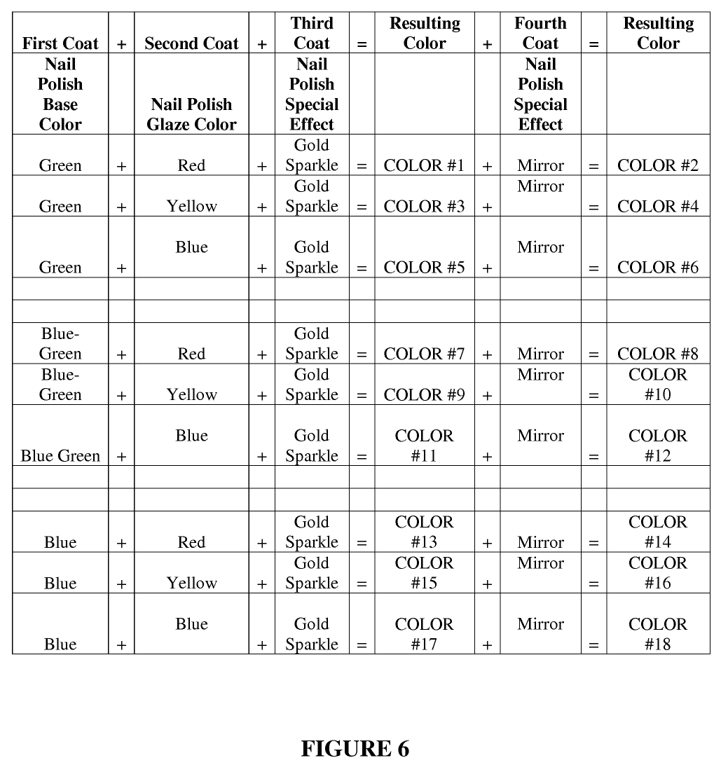

FIG. 6 is an illustration of a color chart of one embodiment of the method of coloring nails and shows the application of two different nail polish special effects. As shown in FIG. 6, a second nail polish special effect, in this case mirror, may be applied immediately after the application of a first nail polish special effect. FIG. 6 also shows the preferred application of a sparkle or glitter nail polish special effect. Specifically, it is preferred that the sparkle nail polish special effect is followed by an application of the mirror (shiny or reflective) nail polish special effect. The mirror nail polish special effect mutes the effect of the sparkle so that it doesn't detract the view from the beautiful colors on which the sparkle is layered.

Preferably, any additional application of a nail polish base, glaze, or special effect is only applied after the previous nail polish is completely dry.

The preferred method of the present invention is the combination of a coat of nail polish base with between one to three coats of nail polish glaze.

In summary, the present invention is a translucent and colored nail polish glaze formulation, a nail polish base formulation, and a method for combining nail polish base colors with translucent and colored nail polish glazes to create a multiple of different colors and color shades from a minimal number of nail polish base and glaze colors.

The foregoing description of the preferred embodiment of the invention has been presented for the purposes of illustration and description. While multiple embodiments are disclosed, still other embodiments of the present invention will become apparent to those skilled in the art from the above detailed description, which shows and describes illustrative embodiments of the invention. As will be realized, the invention is capable of modifications in various obvious aspects, all without departing from the spirit and scope of the present invention. Accordingly, the detailed description is to be regarded as illustrative in nature and not restrictive. Also, although not explicitly recited, one or more embodiments of the invention may be practiced in combination or conjunction with one another. Furthermore, the reference or non-reference to a particular embodiment of the invention shall not be interpreted to limit the scope the invention. It is intended that the scope of the invention not be limited by this detailed description, but by the claims and the equivalents to the claims that are appended hereto.

What is claimed is:

1. A nail polish, comprising:
ethyl acetate;
butyl acetate;
isopropyl alcohol;
nitrocellulose;
stearalkonium hectorite;
diacetone alcohol;
citric acid;
dimethicone; and
benzophenone-1.

2. The nail polish of claim 1, further comprising:
one or more chemicals selected from the group consisting of, adipic acid, neopentyl glycol, trimellitic anhydride copolymer;
trimethyl pentanyl diisobutyrate; and
triphenyl phosphate.

3. The nail polish of claim 2, further comprising:
one or more chemicals selected from the group consisting of: nacreous pigments; red no. 6 barium lake, red no. 7 calcium lake, red no. 34 calcium lake, yellow no. 5 aluminum lake, ferric (ammonium) ferrocyanide, and violet no. 2;
wherein said nail polish is a translucent nail polish glaze.

4. The nail polish of claim 3, further comprising:
one or more chemicals selected from the group consisting of: SD alcohol 40B; SD alcohol 3-A; N-butyl alcohol; camphor; black iron oxide; red iron oxide; titanium dioxide; tosylamide; epoxy resin; styrene acrylates copolymer; silica; polyethylene terephthalate; acrylates copolymers;
wherein said nail polish is a translucent nail polish special effect glaze.

5. The nail polish of claim 2, further comprising:
one or more chemicals selected from the group consisting of: nacreous pigments; stearalkonium hectorite, titanium dioxide, mica, bismuth oxychloride, talc, iron oxides, red no. 6 barium lake, red no. 7 calcium lake, red no. 34 calcium lake, yellow no. 5 aluminum lake, ferric (ammonium) ferrocyanide, and violet no. 2;
wherein said nail polish is an opaque nail polish base.

6. A method of coloring the nails of a human comprising the steps:
selecting a nail polish base color;
applying said nail polish base color to an one or more nails;
selecting a first nail polish glaze color;
applying said first nail polish glaze color to said one or more nails, wherein said first nail polish glaze color is applied only to said one or more nails with said nail polish base color;
wherein said first nail polish glaze color is translucent.

7. The method of coloring the nails of a human of claim 6, further comprising the steps of:
providing an one or more color charts that describes an one or more steps to create an one or more resulting colors that result from combining an one or more nail polish bases with an one or more nail polish glazes and an one or more nail polish special effects;
determining what a final color of said one or more nails will be by consulting said one or more color charts;
following said one or more steps to create said determined final color of said one or more nails;
wherein a combination of said first nail polish glaze color, which covers in a layered manner said nail polish base color on said one or more nails, causes said one or more nails to have a third color that is different from said nail polish glaze color and said nail polish base color.

8. The method of coloring the nails of a human of claim 7, further comprising the step of:
applying said first nail polish glaze color a second time to said one or more nails.

9. The method of coloring the nails of a human of claim 8, wherein said second application of said first nail polish glaze color causes said one or more nails to have a fourth color that is different from said third color, said nail polish glaze color, and said nail polish base color.

10. The method of coloring the nails of a human of claim 9, wherein said fourth color is a deeper color shade than said third color.

11. The method of coloring the nails of a human of claim 7, further comprising the step of:
applying a second nail polish glaze color to said one or more nails;
wherein said second nail polish glaze color is translucent;
wherein said second nail polish glaze color is different from said first nail polish glaze color and is applied after said first nail polish glaze color.

12. The method of coloring the nails of a human of claim 9, further comprising the step of:
applying a first nail polish special effect after applying said nail polish base color and before applying said first nail polish glaze color.

13. The method of coloring the nails of a human of claim 9, further comprising the step of:
applying a first nail polish special effect to said one or more nails after applying said first nail polish glaze color and before applying said second application of said first nail polish glaze color.

14. The method of coloring the nails of a human of claim 13, further comprising the step of:
applying said first nail polish special effect a second time to said one or more nails.

15. The method of coloring the nails of a human of claim 12, further comprising the step of:
applying a second nail polish special effect to said one or more nails after applying said first nail polish special effect.

16. The method of coloring the nails of a human of claim 13, further comprising the step of:
applying a second nail polish special effect to said one or more nails after applying said first nail polish special effect.

17. The method of coloring the nails of a human of claim 11, further comprising the step of:
applying a first nail polish special effect after applying said nail polish base color and before applying said first nail polish glaze color.

18. The method of coloring the nails of a human of claim 11, further comprising the step of:
applying a first nail polish special effect to said one or more nails after applying said first nail polish glaze color and before applying said second nail polish glaze color.
